(19) United States
(12) Patent Application Publication
(10) Pub. No.: US 2008/0081330 A1
(43) Pub. Date: Apr. 3, 2008

(54) Method and devices for analyzing small RNA molecules

(75) Inventor: Avak Kahvejian, Somerville, MA (US)

Correspondence Address: COOLEY GODWARD KRONISH LLP;ATTN: Patent Group, Suite 1100, 777 - 6th Street, NW, WASHINGTON, DC, 20001, US

(73) Assignee: Helicos BioSciences Corporation, Cambridge, MA (US)

(21) Appl. No.: 11/540,617

(22) Filed: Sep. 28, 2006

(51) Int. Cl. C12Q 1/68; C12M 1/34; G01N 33/00; C12P 19/34

(52) U.S. Cl. 435/6; 422/68.1; 435/287.2; 435/91.5; 436/94

(57) ABSTRACT

The instant inventon provides methods and devices for detecting, enumerating and/or identifying small RNA molecules using single molecule sequencing techniques.

### TECHNICAL FIELD OF THE INVENTION

This invention relates to methods, devices, and combination articles of manufacture for detecting, enumerating, and identifying small RNAs. According to the invention, small RNAs are modified with an adaptor such that they can be attached to a surface for sequence analysis.

### BACKGROUND OF THE INVENTION

Small RNAs are repressors of gene expression found ubiquitously in eukaryotes. Small RNAs are typically about 21 to about 26 nucleotides in length and induce repression through homologous sequence interactions. There are many types of small RNAs including short interfering (si)RNAs, small temporal (st)RNAs, heterochromatic siRNAs, tiny non-coding RNAs, and micro (mi)RNAs. Small RNAs can control mRNA stability or translation, or target epigenetic modifications to specific regions of the genome. Small RNAs are typically produced by processing of longer double-stranded RNA precursors by an RNaseIII-like enzyme.

Small RNAs regulate gene expression in a wide range of biological activities from development to host defense pathways against foreign nucleic acids. For example, siRNAs are triggered by transgenes, micro-injected RNA, viruses, and transposons, whereas miRNAs appear to down regulate endogenous genes involved in developmental programs in animals and plants. The expression of many miRNA genes in Arabidopsis, C. elegans, mice and Drosophila is developmentally regulated.

Both miRNAs and siRNAs appear to silence gene expression at the posttranscriptional level. Both appear to act by virtue of their sequence complementarity to target mRNAs. For example, siRNAs associate with an endonuclease-containing complex, causing the degradation of the associated mRNA; a process termed RNAi in animals, post-transcriptional gene silencing (PTGS) in plants, and quelling in the filamental fungus Neurospora crassa. On the other hand, miRNAs can act in two different mechanisms. They can act similarly to siRNAs where the associated mRNA is guided to an endonuclease-containing complex, or they can base pair with the 3 UTR of mRNAs and block translation.

Over 200 miRNAs have been identified from several organisms. However, computational analyses of genomes have revealed that many more miRNAs are likely to exist that have eluded the various cloning strategies to date. There are many reasons why small RNAs have eluded cloning. For example, miRNAs are often expressed in a tissue-specific manner. In addition, miRNAs are often present in low abundance or are expressed during a brief window of time.

Current methods used to identify small RNA molecules include Northern blotting, RNase protection assays, or cloning followed by sequencing. Assays such as Northern blotting and RNase protection require gel electrophoresis. Detection by Northern blotting is problematic because of the low sensitivity of the assay, often requiring microgram quantities of RNA. In addition, the transfer required by Northern blotting often has low reproducibility of RNA to a solid support, required by Northern blotting due to the small size of the RNA target molecules. Furthermore, hybridization may not discriminate between closely related small RNAs. RNase protection assays are less desirable because of the requirement for highly radioactive probes. Cloning of individual small RNAs followed by sequencing is effective in determining single-base differences between closely related small RNAs, however the technique is time consuming and thus far not amenable to high throughput. Therefore, more efficient and accurate methods for detecting, enumerating, and identifying small RNA molecules are needed, in particular, methods that are amenable to high throughput.

### SUMMARY OF THE INVENTION

The invention provides methods, apparatus, and compositions for the detection, enumeration, and identification of small RNA molecules. According to methods of the invention, detection of small RNA molecules is achieved by attaching the modified small RNA to a surface at single molecule resolution, and analyzing the sequence of the attached small RNA molecules. The invention provides sample preparation, attachment strategies, surface preparation, and rinsing strategies that result in improved detection, enumeration, and identification of small RNA molecules in a biological sample.

There is a variety of ways in which small RNA molecules can be identified, sequenced and/or characterized. Each involves placing molecules on a surface such that at least a plurality of them are individually optically resolvable. In an embodiment of the invention, small RNA molecules, or cDNA transcripts of small RNA molecules, are attached directly to a surface that has been treated to minimize background for optical detection of incorporated nucleotides in a template-dependent synthesis reaction conducted on the surface. In one method, small RNA molecules are prepared and attached to an epoxide surface on a glass slide by direct amine attachment at the 5′ end of the nucleic acid. A primer that specifically hybridizes to a portion of the small RNA or cDNA is added. In an alternative embodiment, primers are attached to the surface, and the small RNA to be sequenced is then added for hybridization with the primers. Direct amine attachment to the epoxide surface (described in detail below) secures the small RNA molecule (or primer) to the surface in a manner that is resistant to disruption in wash or nucleotide addition cycles.

In an alternative embodiment of the invention, an attachment sequence is added to the small RNA molecule prior to exposure to the surface. For example, a polynucleotide (e.g., polyadenine) tag is added to the 5′ terminus of the small RNA molecule to be sequenced. A primer containing the complement of the polynucleotide tag is then applied to the surface and is used to capture the polynucleotide tag on the 5′ terminus of the small RNA. The tag can be placed on the 3′ terminus if subsequent sequencing is to proceed toward the surface. The polynucleotide tag can be added enzymatically, by ligation, or by other known techniques.

In one embodiment, single molecule sequencing is combined with hybrid capture. The hybrid capture step is used to select molecules to be sequenced, the captured sequence becoming the duplex for sequencing. Small RNA molecules, or their cDNA complements, can be captured directly or a ligated tag can be the substrate for hybrid capture. For direct capture of a small RNA molecule (e.g., siRNA, miRNA), the melting temperature of the capture duplex must be considered in order to effect proper duplex stability.

Sequencing templates can be direct RNA or a cDNA complement. The cDNA can be prepared in solution using standard methods and conditions or can be prepared on the surface by hybridization to a surface-attached primer that is complementary to the RNA to be copied. Enzymes for use in methods of the invention can be any enzyme capable of catalyzing template-dependent nucleotide addition to a primer. For example, a DNA polymerase or reverse transcriptase enzyme can be used.

According to one embodiment of the invention, the surface comprises a layer of epoxide molecules arranged in a substantially uniform way, for example, substantially in the form of a monolayer. In some embodiments, which may include any of the elements described below, it is advantageous to block non-specific binding sites that may interfere with detection of incorporation events during nucleic acid sequencing reactions. Agents such as water, sulfate, an amine group, a phosphate or a detergent may be used to block non-specific binding. A detergent, such as Tris, can serve to block or passivate the epoxide molecules alone or in conjunction with other blocking agents. Thus, a detergent may be incorporated into surface washing steps in order to preserve a passivated surface and prevent excess background that may interfere with detection. Blocking can occur by exposing the surface to molecules that compete with non-specific binding or that reduce or eliminate the reactive portion of the surface molecule. For example, water can open the epoxide ring, making it less reactive. Thus, after attachment of primers or small RNA molecules, an epoxide surface can be rinsed in order to reduce or eliminate the reactive functionality of the epoxide, thus reducing non-specific binding.

In a preferred embodiment, surfaces are prepared and treated for single molecule sequencing. True single molecule sequencing differs from traditional bulk sequencing, inter alia, in that true single molecule sequencing allows sequencing of individual nucleic acids without amplification. In a typical single molecule reaction, individual nucleotide triphosphates, having an optically-detectable label (e.g., a fluorescent molecule) attached, are added in a template-dependent fashion to the primer portion of a primer/template duplex. Individual incorporated nucleotides are imaged, via their attached labels, upon incorporation and a sequence is compiled based upon the sequential addition of nucleotides to the primer.

A stationary and stable template nucleic acid is preferred for sequencing. As such, various primer/template anchoring methods are used to promote duplex stability. For example, duplex may be stabilized by using nucleic acids that form covalent linkages with their complement, by utilizing nucleic acid binding proteins, or by the use of specific binding partners (e.g., biotin/streptavidin), or by other methods known in the art. In one example, a template sequence is 3′ end-labeled (e.g., with a dideoxy nucleotide) having one member of the binding pair attached. The other member of the binding pair is attached to the surface. Once the duplex is stabilized in this way, sequencing proceeds as described below.

Regardless of whether the small RNA, a cDNA, or a primer is first anchored to the surface, sequencing proceeds upon duplex formation in a template-dependent manner. For example, if a primer of template-dependent synthesis is attached to the surface (e.g., by direct amine attachment), a small RNA having a sequencing complementary to the sequence of the primer is exposed to the surface in order to form a duplex with the primer. After non-complementary sequence is washed off the surface, the remaining duplex are exposed to a polymerase and at least one nucleotide having an optically-detectable label under conditions that are sufficient for addition of a complementary nucleotide to the 3′ terminus of the primer. Complementary nucleic acids are added in a template-dependent manner to those duplex in which the added base is complementary to the 3′ terminal base on the primer. The surface is rinsed in order to remove unincorporated nucleotides and the surface is imaged in order to determine which duplex added a nucleotide (by positional detection of the optical label). Enzyme-mediated, template-dependent nucleotide addition is then repeated until sufficient sequencing information is obtained from a sufficient number of duplex on the surface.

As will be appreciated by one skilled in the art, individual features of the invention may be used separately or in any combination. A detailed description of embodiments of the invention is provided below. Other embodiments of the invention are apparent upon review of the detailed description that follows.

### DETAILED DESCRIPTION OF THE INVENTION

The present invention provides methods for detecting, enumerating, and identifying small RNA molecules from a biological sample without having to amplify the small RNA molecules. Devices are provided for performing the method and combination articles of manufacture are provided for detecting, enumerating, and identifying small RNA molecules according to the method of the invention.

Preferred methods for detecting small RNA molecules in a biological sample comprise modifying small RNA molecules with an adaptor and attaching the modified small RNA molecules to a surface, either directly or via hybridization to a complementary primer on a surface. The small RNA molecules can be obtained from a biological sample. Individual small RNA molecules are positioned on the surface such that they are individually optically resolvable. The attached modified small RNA molecules are analyzed such that at least one nucleotide is identified in at least one attached modified small RNA molecule thereby detecting small RNA molecules in a biological sample. In an optional embodiment, the analyzing step is repeated, and the identified nucleotides compiled in order to determine the entire sequence of at least one small RNA molecule.

In a preferred embodiment, RNA is extracted from a biological sample and then separated by size. RNA corresponding to about 10 to about 200 nucleotides in length is obtained from the separated RNA. The RNA obtained is then modified with an adaptor. The modified RNA is then attached to a surface, wherein individual modified RNA molecules are positioned on the surface such that at least two of the individual modified RNA molecules are individually optically resolvable. The attached modified RNA molecules are then analyzed, wherein at least one nucleotide is identified in at least one attached modified RNA molecule.

### Small RNA

As used herein, small RNA molecules include both miRNA and siRNA. The fractionated RNA molecules of the invention preferably have a length of about 18 to about 100 nucleotides, and more preferably from about 18 to about 80 nucleotides. Mature small RNAs usually have a length of 19-26 nucleotides, particularly 21, 22 or 23 nucleotides. The small RNA may also be provided as a precursor, which usually has a length of 50-90 nucleotides, particularly 60-80 nucleotides. Precursors may be produced by processing a primary transcript which may have a length of greater than 100 nucleotides.

The small RNA molecules may be single-stranded, double-stranded, or double-stranded with single-stranded regions. For example, miRNA is usually a single-stranded molecule, while the miRNA-precursor is usually an at least partially self-complementary molecule capable of forming double-stranded portions (e.g. stem- and loop-structures).

Small RNA molecules can be obtained from any cell of a person, animal, plant, or virus, or any other cellular organism. RNA can be prepared from any suitable biological sample that contains or is expected to contain small RNA molecules (tissue samples, whole organisms, cell cultures, bodily fluids). Small RNA molecules may be obtained directly from an organism or from a biological sample from an organism, e.g., from blood, urine, cerebrospinal fluid, seminal fluid, saliva, sputum, stool and tissue. Any tissue or body fluid specimen may be used according to methods of the invention. Small RNA molecules may also be isolated from cultured cells, such as primary cell culture or cell lines of a given organism.

Many methods are available for the isolation and purification of small RNA molecules for use in the present invention. Preferably, the small RNA molecules are sufficiently free of proteins and any other interfering substances to allow specific primer annealing and extension.

The preparation of RNA preferably involves removal of most or all other biomolecules. Protein and DNA are generally removed from the preparation. Lipids and carbohydrates can usually be removed from the preparation with the aid of a detergent. Protein can be removed with the aid of detergents, denaturants and/or enzymes that degrade proteins, such as ProteinaseK.

Total RNA can be prepared from biological samples by methods well known in the art. For example, using methods described in U.S. Patent Application 2005/0059024 A1, published Mar. 17, 2005, the teachings of which are incorporated herein in their entirety. In one method, cells of a biological sample are lysed by suitable methods. Organic solvents that are immiscible with water are used to extract proteins by precipitation. The aqueous, protein-free phase is separated by centrifugation and removed. Usually, phenol or phenol-chloroform mixtures are used for this purpose. Phenol and phenol-chloroform extractions provide an extremely protein- and lipid-free solution of nucleic acid. Much if not all (depending on the sample) of the carbohydrate is also lost in this procedure as well. Acid phenol-chloroform is known to extract some of the DNA out of the aqueous solution. However, the solution is high in denaturing agents such as guanidinium hydrochloride, guanidinium thiocyanate, or urea, all of which interfere with downstream enzymatic analysis, while guanidinium compounds interfere with electrophoretic analysis. The denaturing agents can be removed from the RNA extract prior to fractionation. RNA is usually separated from these mixtures by selective precipitation, usually with ethanol or isopropanol.

In another method, RNA from lysed cells is selectively immobilized on a solid surface and any protein is rinsed away. The RNA is then released under suitable conditions. Both procedures can reduce the amount of DNA contamination or carryover, with the efficiency varying according to the precise conditions employed.

A third method involves isolating small RNA molecules from cells comprising: a) lysing the cells with a lysing solution to produce a lysate; b) adding an alcohol solution to the lysate; c) applying the lysate to a solid support; and d) eluting RNA molecules having the desired length from the solid support, according to U.S. Patent Application No. 2005/0059024 A1.

To obtain total RNA, a biological sample containing cells is lysed or homogenized to produce a lysate. A lysing solution including a chaotropic agent or detergent is preferably used. A chaotropic agent can be any agent that unfolds ordered macromolecules, thereby causing them to lose their function (hence causing binding proteins to release their target). A detergent can be any substance that can disperse a hydrophobic substance (usually lipids) in water by emulsification (e.g., SDS).

Homogenization or lysing of a cell can be accomplished using a solution that contains a guanidinium salt, detergent, surfactant, or other denaturant. The terms homogenization and lysing are used interchangeably. The concentration of a chaotropic agent in the solutions of the invention, particularly lysing solutions, is about 0.5 to about 5 M. For example, the concentration of guanidinium in the lysing solution is between about 2.0 M and 3.5 M. Guanidinium salts are well known to those of skill in the art and include guanidinium hydrochloride and guanidinium isothiocyanate. Additionally, a homogenization solution may contain urea or other denaturants, such as NaI.

A biological sample may be homogenized or fractionated in the presence of a detergent or surfactant. The detergent can act to solubilize the sample. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Tritin® X-100R, Triton® X-114 x=7-8, octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid.

The buffer is at a concentration of about 5 to about 500 mM in the solution or in the solution with the sample. In a preferred embodiment, the buffer concentration in the lysing solution is between about 10 mM and 300 mM. The buffer can be, for example, TrisCl, other buffers suitable for lysing cells of a biological sample may be used as well.

In a preferred embodiment, the lysis solution includes: guanidinium thiocyanate, N-lauroyl sarcosine, and TrisHCl. Once the sample has been homogenized in the lysing solution, the RNA can be extracted, often with phenol solutions or the use of an adsorptive solid phase. Alternative methods use combination denaturant/phenol solutions to perform the initial homogenization, precluding the need for a secondary extraction. Examples of these reagents would be Trizol™ (Invitrogen) or RNAwiz™ (Ambion, Inc.)

Subsequent to exposure to a homogenization solution, samples may be further homogenized by mechanical means. Mechanical blenders, rotor-stator homogenizers, or shear-type homogenizers may be employed. Alternatively, the tissue could be homogenized in the lysis solution, and the tissue remains separated by settling, centrifugation, or filtration. These remains could then be treated with homogenization solution and extraction conditions as described above.

After lysing the cells in the lysing buffer, an alcohol solution is added to the lysate. The alcohol solution contains at least one alcohol and can be about 5 to about 100% alcohol. The alcohol solution is added to a lysate to make the resulting solution have a concentration of alcohol of about 5 to about 90%. Alcohols include, but are not limited to, ethanol, propanol, isopropanol, and methanol. An alcohol solution may be used in additional steps of methods of the invention to precipitate RNA. The pH of any solution, or of the buffer component of any solution, or of any solution with the sample is preferably between about 4.5 and 10.5.

Small RNA molecules can be extracted from the lysate with an extraction solution comprising a non-alcohol organic solvent prior to applying the lysate to the solid support. The extraction solution contains a non-alcohol organic solvent such as phenol and/or chloroform. The non-alcohol organic solvent solution contains at least one non-alcohol organic solvent, though it may also contain an alcohol. The concentrations described above with respect to alcohol solutions are applicable to concentrations of solutions having non-alcohol organic solvents. In specific embodiments, equal amounts of the lysate and phenol and/or chloroform are mixed. In specific embodiments, the alcohol solution is added to the lysate before extraction with a non-alcohol organic solvent.

Small RNA can be obtained from the total RNA obtained from the lysate by fractionating the RNA on a polyacrylamide gel using standard methods for fractionating small nucleic acids. RNA having the desired size is extracted from the gel and modified with an adaptor as described herein. Small RNA molecules can also be isolated using a solid support, such as a mineral or polymer support as described in U.S. Patent Application 2005/0059024 A1, published Mar. 17, 2005, the teachings of which are incorporated herein in their entirety. RNA corresponding to about 10 to about 200 nucleotides can be obtained. RNA corresponding to no more than about 100, no more than about 50, or no more than about 25 nucleotides in length can be obtained.

### Modifying Small RNA With an Adaptor

As described herein, RNA molecules or small RNA molecules obtained as described herein can be modified by the addition of an adaptor or attachment sequence comprising a specific sequence. Typically, the specific sequence is a homopolymer, such as oligo(dA), and the corresponding primer includes an oligo(dT) sequence. The specific sequence oligonucleotide and primer are chosen such that the modified small RNA can hybridize to the primer. The sequence specific oligonucleotide is of a length suitable for hybridizing a primer for sequencing the small RNA. The oligonucleotide can be about 10 to about 50 nucleotides in length. It is routine in the art to adjust primer length and/or oligonucleotide length to optimize sequencing.

In one embodiment, a universal primer is tethered to a surface, and the template (e.g., small RNA molecule) is modified with an adaptor comprising an oligonucleotide sequence that is complementary to the universal primer, thereby allowing the template to hybridize to the immobilized primer. In another embodiment, the adaptor contains an oligonucleotide sequence and a linker moiety that allows the modified small RNA molecule to be tethered to the surface. Where the template includes the primer complementary oligonucleotide sequence, the adaptor can comprise a linker moiety.

The adaptor can be attached to the RNA or small RNA molecule with an enzyme. The enzyme can be a ligase or a polymerase. The ligase can be any enzyme capable of ligating an oligonucleotide (RNA or DNA) to the DNA or small RNA molecule. Suitable ligases include T4 DNA ligase and T4 RNA ligase (such ligases are available commercially, from New England Biolabs, (on the World Wide Web at NEB.com). In a preferred embodiment, the RNA or small RNA molecules are dephoyshorylated before ligating the adaptors. Methods for using ligases are well known in the art. The polymerase can be any enzyme capable of adding nucleotides to the 3′ terminus of small RNA molecules. The polymerase can be, for example, yeast poly(A) polymerase, commercially available from USB (on the World Wide Web at USBweb.com). The polymerase is used according to the manufacturer's instructions.

### Attaching Modified RNA or Small RNA to a Surface

Generally, a surface or substrate of the present invention may be of any suitable material that allows RNA or small RNA molecules to be individually optically resolvable. Substrates for use according to the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Surfaces suitable for the present invention also include three-dimensional substrates such as, for example, spheres, tubes (e.g., capillary tubes), microwells, microfluidic devices, filters, or any other structure suitable for anchoring a nucleic acid. For example, a substrate can be a microparticle, a bead, a membrane, a slide, a plate, a micromachined chip, and the like. Substrates can include planar arrays or matrices capable of having regions that include populations of target nucleic acids or primers. Examples include nucleoside-derivatized CPG and polystyrene slides; derivatized magnetic slides; polystyrene grafted with polyethylene glycol, and the like.

In one embodiment, a substrate comprises a suitable material that allows for single molecules to be individually optically resolvable. For example, the detection limit can be in the order of a micron. This implies that two molecules can be a few microns apart and be resolved, that is individually detected and/or detectably distinguished from each other. Factors for selecting substrates include, for example, the material, porosity, size, and shape. Substrates that can lower (or increase) steric hindrance of polymerase are preferred. Other important factors to be considered in selecting appropriate substrates include size uniformity, efficiency as a synthesis support, and the substrate's optical properties, e.g., clear smooth substrates (free from defects) provide instrumentational advantages when detecting incorporation of nucleotides in single molecules (e.g., primers hybridized to small RNA molecules).

Preferably, a substrate used according to the invention includes a biocompatible or biologically inert material that is transparent to light and optically flat (e.g., with a minimal micro-roughness rating). Specially manufactured, or chemically derivatized, low background fluorescence substrates (e.g., glass slides) are also contemplated according to the invention. Substrates may be prepared and analyzed on either the top or bottom surface of the planar substrate (i.e., relative to the orientation of the substrate in the detection system.) In addition, a substrate should have minimal defects that are responsible for the production of background that might interfere with detection of incorporated nucleotides. As such, a substrate can be pre-treated with a biocompatible or biologically inert material that creates a planar surface free from defects prior to use in the attachment and/or sequencing methods discussed herein.

Surfaces can be treated to remove defects that are responsible for the production of background that can interfere with detection of surface chemical events (e.g., incorporation of nucleotides). As such, surfaces can be treated, associated or chemically modified with one or more coatings or films that increase binding affinity or improve localization of the bound reactants. Increased surface binding affinity also leads to increased surface retention, maximizing the availability of reactants on the surface. Exemplary films or coatings include epoxides, including those that are derivatized (e.g., with a binding molecule, such as streptavidin).

As discussed herein, not only can a surface be treated to remove defects that are responsible for the production of background, a surface can be treated to improve the positioning of attached molecules, such as primers or small RNA molecules, for analysis. As such, a surface according to the invention can be treated with one or more charge layers (e.g., a negative charge) to repel a charged molecule (e.g., a negatively charged labeled nucleotide). For example, a substrate according to the invention can be treated with polyallylamine followed by polyacrylic acid to form a polyelectrolyte multilayer. The carboxyl groups of the polyacrylic acid layer are negatively charged and thus repel negatively charged labeled nucleotides, improving the positioning of the label for detection.

In some embodiments, the substrates (e.g., glass slides) are associated or derivatized with one or more coatings and/or films that increase molecule-to-substrate binding affinity (e.g., primer or small RNA molecule-to-glass). Increased molecule-to-substrate binding affinity results in increased molecule retention during the various stages of substrate preparation and analysis (e.g., hybridization, staining, washing, scanning stages, and the like, of preparation and analysis). Additionally, in preferred embodiments, coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings and films include, vapor phase coatings of 3-aminopropyltrimethoxysilane, as applied to glass slide products, for example, from Molecular Dynamics, Sunnyvale, Calif. In addition, generally, hydrophobic substrate coatings and films aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase target molecule binding to the substrate or, at least, do not substantially impair target binding.

Other approaches to coat or film substrates comprise associating chemical agents to the substrate, whereby the coating or film is selected for their reactivity with molecules or nucleic acid targets. For example, organo-amine and organo-aldehyde reactive groups at a concentration of about 5×10¹² reactive groups/cm², for example, can be applied to a substrate. These reactive groups increase the binding affinity of nucleic acids, proteins, small molecules, extracts, and whole or fragmented cells, etc. to substrates. Substrate coatings and films are preferentially applied as monolayers, however more than one layer can be applied as appropriate. In some embodiments of the present invention, the substrates are fabricated using photolithographic technologies. Maskless substrate fabrication technology is also known in the art.

Attachment of nucleic acids (e.g., primers or small RNA molecules) to the surface can be by either direct or indirect means. For example, the 5′ end of the adaptor, RNA, or small RNA molecule may be modified to carry a linker moiety for tethering the RNA to the substrate. Alternatively, the 5′ end of the primer may be modified to carry a linker moiety for tethering the primer to a substrate. The RNA or small RNA molecule containing primer complementary oligonucleotide sequence is then immobilized on the surface by hybridizing to the immobilized primer. Methods for immobilizing nucleic acid on a surface of a substrate are described in detail herein and are well known to those of skill in the art and will vary depending on the solid phase support chosen. For example, on an epoxide surface, attachment is either via direct attachment through a reactive amino addition or indirect attachment via a bi-functional bridge. A preferred means of indirect attachment is via a biotin-streptavidin linkage.

Various methods can be used to anchor or immobilize the nucleic acids (e.g., primer, RNA, or small RNA molecules) to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mole. Bio. Rep. 11:107-115, 1986. The bonding also can be through non-covalent linkage. For example, biotin-streptavidin (Taylor et al., J. Phys. D. Appl. Phys. 24:1443, 1991) and digoxigenin with anti-digoxigenin (Smith et al. Science 253:1122, 1992) are common tools for anchoring nucleic acids to surfaces and parallels. Alternatively, the attachment can be achieved by anchoring a hydrophobic chain into a lipidic monolayer or bilayer. Other methods for known in the art for attaching nucleic acids to supports also can be used.

In one aspect, preferred embodiments of the invention include the use of a surface that comprises an epoxide. An epoxide is an ether in which oxygen is part of a three-member ring structure that is under conformational strain. An epoxide is a more reactive than other ethers due to the strained ring structure.

In a preferred embodiment, the surface of a substrate is coated with an epoxide monolayer. An epoxide monolayer may be deposited onto a surface by many methods known in the art, including silanization. Different molecules or combinations of molecules may serve to link the epoxide to a surface. Ideally, a surface will be coated with an even distribution of epoxides prior to nucleic acid (e.g., primer RNA, or small RNA molecules) introduction. When using epoxide, it is important that the surface be comprised mainly of unreacted epoxide. Reacted epoxide will form an alcohol on the surface which will have difficulty reacting with linker molecules.

For example, a nucleic acid (e.g., primer, RNA, or small RNA molecules) can be directly or indirectly linked to an epoxide on the surface of a substrate. In a direct attachment embodiment, the epoxide is introduced to a nucleic acid bearing an amine group. The highly-reactive epoxide ring opens, and a reactive carbon binds to the amine group on the template.

Nucleic acid (e.g., primer, RNA, or small RNA molecules) can also be indirectly linked to an epoxide on the surface of a substrate. When biotin-streptavidin linkage is used to anchor the nucleic acids, the nucleic acids can be biotinylated, while one surface of the substrates can be coated with streptavidin. Since streptavidin is a tetramer, it has four biotin binding sites per molecule. Thus, it can provide linkage between the surface and the biotinylated nucleic acid. The nucleic acid is linked to an epoxide that has been exposed to a biotinylated amine. Upon exposure, the amine reacts with the epoxide ring, and therefore, links the biotin to the epoxide. The biotinylated epoxide is further exposed to streptavidin to coat the substrate. A biotinylated nucleic acid template then is introduced to the substrate. (See, Taylor et al., J. Phys. D. Appl Phys. 24:1443, 1991).

Such treatment leads to a high density of streptavidin on the surface of the substrate allowing a correspondingly high density of template coverage. Surface density of the nucleic acid molecules can be controlled by adjusting the concentration of the nucleic acids applied to the surface. Reagents for biotinylating a surface can be obtained, for example, from Vector Laboratories. Alternatively, biotinylation can be performed with BLCPA: EZ-Link Biotin LC-PEO-Amine (Pierce, on the World Wide Web at Piercenet.com), or any other known or convenient method.

In some embodiments, labeled streptavidin of very low concentration (e.g., in the μM, nM or pM range) is used to coat the substrate surface prior to anchoring. This can facilitate immobilization of the nucleic acid with single molecule resolution. It also can allow detecting spots on the substrate to determine where the nucleic acid molecules are attached, and to monitor subsequent nucleotide incorporation events. Blocking of any unbound epoxide on the surface can be accomplished using any of the methods according to the invention described herein.

Other examples of linkers include antigen/antibody, digoxigenin/anti-digoxigenin, dinitrophenol, fluorescein, and other haptens known in the art. Alternatively, the nucleic acid may contain other binding moieties that result in a conformational change of the epoxide ring and result in a direct attachment of the template to the opened epoxide ring.

Unfortunately, the same properties that make the epoxide reactive to an amine group on the nucleic acid (e.g., primer, RNA, or small RNA molecules) also make the epoxide reactive to other molecules, thereby increasing the likelihood of non-specific binding. In order to inhibit non-specific binding of molecules to a surface comprising an epoxide during nucleic acid sequencing reaction, epoxides not bound to nucleic acid should be passivated (blocked).

Functionalized surfaces for oligonucleotide attachment also are contemplated by the invention. For example, functionalized silicon surfaces are prepared by UV-mediated attachment of alkenes to the surface. UV light mediates the reaction of t-butyloxycarbonyl (t-BOC) protected omega-unsaturated aminoalkane (10-aminodec-1-ene) with hydrogen-terminated silicon. Removal of the t-BOC protecting group yields an aminodecane-modified silicon surface. Nucleic acid (e.g., primers or small RNA molecules) is attached to the functionalized surface by coupling the amino groups to thiol-modified oligodeoxyribonucleotides using a heterobifunctional crosslinker. The surface density of nucleic acid may be controlled by adjusting the amount of aminoalkane used. A linear relationship between the mole fraction of aminodecen and the density of hybridization sites has been found. Alternatively, less than all the t-BOC protecting groups are removed prior to nucleic acid exposure.

Preferred blocking strategies include exposing the surface to a non-detectable molecule that adheres to the surface or changes the chemical properties of the surface such that non-specific binding is reduced. In methods in which optically-detectable labels are used, one way to block or passivate the surface is to expose the surface to unlabeled molecules of the same type as those that are labeled. The unlabeled molecules will out-compete labeled molecules for non-specific binding on the surface, thus reducing background due to non-specific label. Other strategies involve treating the surface with phosphate, Tris, a sulfate, or an amine that interacts with the surface to prevent non-specific binding. Non-reactive proteins are also appropriate. In a preferred embodiment, a matrix of blocking reagents is provided on the surface in order to provide a highly washable, low non-specific background surface. In some embodiments, blocking reagents are chosen to provide electrostatic repulsion of highly anionic nucleoside triphosphates.

Any molecule capable of interacting with or breaking the epoxide ring, or binding to available carbons in an already-broken epoxide ring, may be appropriate as a passivating (blocking) agent. A preferred passivating agent should not interfere with intended surface chemistry (e.g., incorporation of a nucleotide or determining/detecting the incorporated nucleotide.) Examples of preferred blocking agents are water, a sulfate group, an amine group, a phosphate (PO₄) or a detergent (such as Tris). Blocking agents may be introduced or reintroduced at any time during the analysis. Also, in some embodiments, blocking agents may be used to pre-treat the surface of the substrate prior to exposing the substrate to a nucleic acid. In addition, blocking agents, such as a detergent (e.g., Tris) may be included in some or all wash steps in order to passivate the surface during incubation periods and/or washes.

Surface charge affects the surface stability of the nucleic acid (e.g., primer, RNA, or small RNA molecules). The effectiveness of performing substrate-based sequencing in general, and single molecule sequencing in particular, depends in part on the conformation of the nucleic acid template on the substrate. During a sequencing reaction, for example, the steric conformation of the nucleic acid template is an important factor for successful primer annealing and primer extension. Although a negatively charged nucleic acid template molecule tends to repel from a negatively charged substrate thereby making attachment of the nucleic acid template to the surface of the substrate more difficult. Once a nucleic acid template is bound to the surface of a substrate, a negative charge on the substrate promotes the proper conformation of the nucleic acid for sequencing purposes. Namely, a negatively charged surface helps repel the nucleic acid template from the surface, projecting the template away from the surface (or substantially orthogonal to a horizontal surface) and making the nucleic acid template more available to reagents such as a primer, polymerase and/or nucleotides (labeled or unlabeled).

As a result, surface charge can be manipulated to achieve ideal conditions during both nucleic acid attachment and primer extension. For example, during the loading phase where the nucleic acid is bound or positioned on the surface, the salt concentration of the solution may be increased in order to create a more positive surface charge on the substrate to facilitate reaction between the amine portion of the nucleic acid and the epoxide ring. Conversely, after the nucleic acid has been secured to the surface, the salt concentration of the solution can lowered in order to repel the nucleic acid nucleic acid from the surface of the substrate thereby sterically conforming the nucleic acid strand for annealing and sequence analysis.

In another embodiment, the substrate includes a layer of polyanions and nucleic acid molecules anchored on the layer of polyanions. Accordingly, nucleic acid molecules are positioned to avoid being substantially parallel (e.g., is hindered from lying down on the layer of polyanions.) In some embodiments, the surface of a substrate is pretreated to create a surface chemistry that facilitates nucleic acid molecule attachment and subsequent sequence analysis. In some of these embodiments, the substrate surface is coated with a polyelectrolyte multilayer (PEM). In some cases, biotin can be applied to the PEM, followed by application of streptavidin. The substrate can then be used to attach biotinylated nucleic acids.

The PEM-coated substrate provides substantial advantages for nucleic acid sequence determination and for polymerization reactions. First, a PEM can easily be terminated with polymers bearing carboxylic acids, thereby facilitating nucleic acid attachment. Second, the attached nucleic acid molecule is available for extension by polymerases due to the repulsion of like charges between the negative carboxylic groups. Also, the negative nucleic acid backbone hinders the nucleic acid molecule from a formation that is substantially parallel to the surface of the substrate. In addition, the negative charges repel unincorporated nucleotides, thereby reducing nonspecific binding and hence background interference.

In some embodiments, multiple layers of alternating positive and negative charges are used. In the case of incompletely-charged surfaces, multiple-layer deposition tends to increase surface charge to a well-defined and stable level. For example, surfaces can be coated with a PEM for attachment of target nucleic acids and/or primers via light-directed spatial attachment. Alternatively, nucleic acids (e.g., primers, RNA, or small RNA molecules) can be attached to a PEM-coated surface chemically. PEM formation has been described in Decher et al (Thin Solid Films, 210:831-835, 1992), the teachings of which are incorporated herein. PEM formation proceeds by the sequential addition of polycations and polyanions, which are polymers with many positive or negative charges, respectively. Upon addition of a polycation to a negatively-charged surface, the polycation deposits on the surface, forming a thin polymer layer and reversing the surface charge. Similarly, a polyanion deposited on a positively charged surface forms a thin layer of polymer and leaves a negatively charged surface. Alternating exposure to poly(+) and poly(−) generates a polyelectrolyte multilayer structure with a surface charge determined by the last polyelectrolyte added. This can produce a strongly-negatively-charged surface, repelling the negatively-charged nucleotides.

Detailed procedures for coating a substrate with PEM for immobilizing nucleic acid are described below. In general, the surface of the substrate (e.g., a glass cover slip) can be cleaned with a RCA solution. After cleaning, the substrate can be coated with a PEM, terminating with carboxylic acid groups. Following biotinylation of the carboxylic acid groups, streptavidin can be applied to generate a surface capable of capturing biotinylated molecules. Biotinylated nucleic acid (e.g., primer, RNA, or small RNA molecules) or primers can then be added to the coated substrate for anchoring. During the immobilization or anchoring step, a high concentration of cation, e.g., Mg²⁺, can be used to screen the electrostatic repulsion between the negatively-charged nucleic acid molecules and the negatively-charged PEM surface. In subsequent steps, the action concentration can be reduced to re-activate repulsive shielding. By titrating biotinylated nucleic acid molecules, it is possible to bind such a small number of molecules to the surface that they are separated by more than the diffraction limit of optical instruments and thus able to be visualized individually.

The attachment scheme described here can be readily generalized. Without modification, the PEM/biotin/streptavidin surface produced can be used to capture or immobilize any biotinylated molecule. A slight modification can be the use of another capture pair, for example, substituting digoxygenin (dig) for biotin and labeling the molecule to be anchored with anti-digoxygenin (anti-dig), or dinitrophenol and its antibody can be used. Reagents for biotinylation or dig-labeling of amines are both commercially available.

Attachment chemistry is nearly independent of the underlying surface chemistry and so permits further generalization. Glass, for instance, can support PEMs terminated with either positive or negative polymers, and a wide variety of chemistry is available for either. But other substrates such as silicone, polystyrene, polycarbonate, etc. or even membranes and/or gels, which are not as strongly charged as glass, can still support PEMs. The charge of the final layer of PEMs on weakly-charged surfaces becomes as high as that of PEMs on strongly-charged surfaces, as long as the PEM has a sufficient number of layers. Thus, advantages of the glass/PEM/biotin/streptavidin/biotin-nucleic acid surface chemistry can readily be applied to other substrates. In some embodiments, the attachment schemes can be either ex-situ or in-situ.

In another aspect of the invention, the substrate may be prepared by, for example, coating with a chemical that increases or decreases hydrophobicity or coating with a chemical that allows covalent linkage of the nucleic acid molecules or other polymeric sequences. Some chemical coatings may both alter the hydrophobicity and allow covalent linkage. Hydrophobicity on a solid substrate may readily be increased by silane treatment or other treatments known in the art. Linker molecules adhere to the surface and comprise a functional moiety that reacts with biomolecules. Many such linkers are readily available and known in the art. For example, substrates or supports are modified with photolabile-protected hydroxyl groups, alkoxy or aliphatic derivatized hydroxyl groups, or other chemicals.

A preferred coating that both decreases hydrophobicity and provides linkers is poly(ethyleneimine). In addition, poly(ethyleneimine) (PEI) coated solid substrates also have the added benefit of long shelf life stability. The coating of silicon wafers and glass slides with polymers such as poly(ethyleneimine) can be performed in-house or through companies such as Cel Associates (Houston, Tex.). Glass slides also can be coated with a reflective material or coated with PEI using silane chemistry. The PEI coating permits the covalent attachment of single or double stranded nucleic acids, single or double stranded long DNA molecules or fragments or any other amine-containing biomolecules to the substrate or support. Nucleic acids may be covalently attached at the 5′ using a hexylamine modification, which places a primary amine at the 5′-end of the nucleic acid. The 5′-amine on the nucleic acid may then be reacted with a cross-linker, such that the nucleic acid is covalently attached to the polymer coating on the solid support.

Methods of the invention also optionally include a surface drying step. In some embodiments, the surface is exposed to a drying agent prior to, during and/or after a chemical reaction, such as a nucleotide incorporation step. Examples of preferred drying agents include, without limitation, phosphate buffer, an alcohol (such as, for example, EtOH), air and/or N₂.

### Analyzing the Attached Small RNA

Modified small RNA molecules can be directly or indirectly immobilized on the surface of a substrate (e.g., a glass or plastic slide, a nylon membrane, or gel matrix) as described herein.

At least one modified small RNA molecule is hybridized to a primer to form a template/primer complex on the surface. Thereafter, primer extension is conducted to identify at least one nucleotide of the hybridized small RNA molecule using a nucleotide polymerizing enzyme and a nucleotide (e.g., dATP, dTTP, dUTP, dCTP and/or a dGTP) or a nucleotide analog. Incorporation of a nucleotide or a nucleotide analog is detected at discrete locations on the surface. Template/primer complex, as well as incorporated nucleotides, are individually resolvable in single molecule embodiments. Alternatively, bulk signal from mixed nucleic acid populations or clonal populations of small RNA molecules, are obtained.

Fast reagent application and removal is another advantage according to the invention. For example, concentrations of nucleotides and/or other reaction reagents can be alternated at different time points of the analysis. This is a particularly useful feature in an embodiment comprising introducing one or more single species of nucleotide individually. This could lead to increased incorporation rates and sensitivity. For example, when all four types of nucleotides are simultaneously present in the reaction to monitor dynamic incorporation of nucleotides, concentrations of the each of the respective nucleotides can be alternated between a first and a second range. This leads to both better visualization of the signals when low concentrations of nucleotides are present, and increased polymerization rate when higher concentrations of nucleotides are present.

Certain embodiments of the present invention avoid many of the problems observed with other sequencing methods. For example, the methods provided herein are highly parallel because many molecules can be analyzed simultaneously at high density (e.g., 1 or 2 million molecules per cm²). Thus, many different small RNA molecules can be sequenced or analyzed on a single substrate surface simultaneously according to methods and devices of the present invention.

Conditions for hybridizing primers to nucleic acid targets (e.g., small RNA molecules) are well known. The annealing reaction is performed under conditions which are stringent enough to guarantee sequence specificity, yet sufficiently permissive to allow formation of stable hybrids at an acceptable rate. The temperature and length of time required for primer annealing depend upon several factors including the base composition, length and concentration of the primer, and the nature of the solvent used, e.g., the concentration of cosolvents such as DMSO (dimethylsulfoxide), formamide, or glycerol, and counterions such as magnesium. Typically, hybridization (annealing) between primers and target nucleic acids is carried out at a temperature that is approximately 5 to 10° C. below the melting temperature of the target-primer hybrid in the annealing solvent. Typically, the annealing temperature is in the range of 55 to 75° C. and the primer concentration is approximately 0.2 μM. Under such conditions, the annealing reaction is usually complete within a few seconds.

Methods according to the invention include conducting a primer extension reaction, such as exposing the target nucleic acid to a primer under conditions sufficient to extend a nucleic acid by at least one base. Sequencing, as used herein can be performed such that one or more nucleotides are identified in one or more small RNA molecules. Methods according to the invention also include the step of compiling a sequence of the molecule (nucleic acid) based upon sequential incorporation of the extension bases into the primer.

In the analyzing step, the attached, modified, small RNA molecules can be sequenced using single molecule sequencing as described, for example, in U.S. patent application Ser. No. 11/137,928, filed May 25, 2005 and/or as described in U.S. Pat. No. 6,780,591, the teachings of both of which are incorporated herein in their entirety. In one embodiment, reverse transcriptase, which catalyzes the synthesis of single-stranded DNA from an RNA template is used as the template-dependent, nucleotide polymerizing enzyme. The RNA template annealed to a primer (template/primer complex) is contacted with dNTPs in the presence of reverse transcriptase enzyme under conditions such that the polymerase catalyzes template-dependent addition of a dNTP to the 3′ terminus of the primer that is complementary to the corresponding nucleotide in the RNA template. The dNTP is detectably labeled, as described herein, and the nucleotide is identified by detecting the presence of the incorporated labeled nucleotide. As described herein, unincorporated labeled dNTPs can be removed (e.g., by washing) from the surface prior to detecting the incorporated labeled dNTP. The process can be repeated one or more times, wherein the RNA template/primer complex(s) are provided with additional dNTPs, in the presence of a reverse transcriptase, followed by removing the unincorporated labeled dNTPs and detecting the incorporated labeled dNTP. The sequence of the RNA is determined by compiling the identified dNTPs. In this manner, the entire sequence of one or more small RNA molecules can be determined. In addition, by using single molecule sequencing techniques, determining the sequence for each small RNA molecule attached to the surface, provides the number of different or unique small RNA molecules in a biological sample. Furthermore, the number of copies of each unique small RNA sequences in a biological sample is also provided.

In order to allow for further extension and detection of subsequently added fluorophore-labeled nucleotides, the fluorophore of the incorporated nucleotide can be destroyed by photochemical destruction as described in U.S. Pat. No. 6,780,591, the teachings of which are incorporated herein in their entirety. This cycle can be repeated a large number of times if sample losses are avoided. In one embodiment, such losses will be avoided by attaching the primer or template strands to a surface of an array device, for example a microscope slide, and transferring the entire array device between a reaction vessel and the fluorescent reader.

In a preferred embodiment, after detection, the label is rendered undetectable by removing the label from the nucleotide or extended primer, neutralizing the label, or masking the label. In certain embodiments, methods according to the invention provide for neutralizing a label by photobleaching. This is accomplished by focusing a laser with a short laser pulse, for example, for a short duration of time with increasing laser intensity. In other embodiments, a label is removed from its nucleotide by photocleavage. For example, a light-sensitive label bound to a nucleotide is photocleaved by focusing a particular wavelength of light on the label. Generally, it may be preferable to use lasers having differing wavelengths for exciting and photocleaving. Labels also can be chemically cleaved. Labels may be removed from a substrate using reagents, such as NaOH, dithiothreitol, or other appropriate buffer reagent. The use of disulfide linkers to attach the label to the nucleotide are especially useful and are known in the art.

The extension reactions are carried out in buffer solutions which contain the appropriate concentrations of salts, dNTP(s) and nucleotide polymerizing enzyme such as reverse transcriptase required for enzyme mediated extension to proceed. For guidance regarding such conditions see, for example, Sambrook et al. (1989, Molecular Cloning, A Laboratory Manual, Cold Spring Harbor Press, NY); and Ausubel et al. (1989, Current Protocols in Molecular Biology, Green Publishing Associates and Wiley Interscience, NY).

Typically, buffer containing one of the four dNTPs is added into the primer/template complexes. Depending on the identity of the nucleoside base at the next unpaired template site in the primer/template complex, a reaction will occur when the appropriate dNTP is present. When any one of the other three incorrect dNTPs is present, no reaction will take place.

In a preferred embodiment of the invention, the primer/template complexes comprise the modified small RNA molecules tethered to a surface to permit the sequential addition of sequencing reaction reagents without complicated and time consuming purification steps following each extension reaction.

The sequencing can be optimized to achieve rapid and complete addition of the correct nucleotide to primers in primer/template complexes, while limiting the misincorporation of incorrect nucleotides. For example, dNTP concentrations may be lowered to reduce misincorporation of incorrect nucleotides into the primer. Kₘ values for incorrect dNTPs can be as much as 1000-fold higher than for correct nucleotides, indicating that a reduction in dNTP concentrations can reduce the rate of misincorporation of nucleotides. Thus, in a preferred embodiment of the invention the concentration of dNTPs in the sequencing reactions are approximately 5-20 μM.

In addition, relatively short reaction times can be used to reduce the probability of misincorporation. For an incorporation rate approaching the maximum rate of about 400 nucleotides per second, a reaction time of approximately 25 milliseconds will be sufficient to ensure extension of 99.99% of primer strands.

While different nucleic acids can be each immobilized to and analyzed on a separate substrate, multiple nucleic acids also can be analyzed on a single substrate. In the latter scenario, the nucleic acids can be bound to different locations on the substrate. This can be accomplished by a variety of different methods, including hybridization of primer capture sequences to nucleic acids immobilized at different locations on the substrate.

In certain embodiments, different nucleic acids also can be attached to the surface of a substrate randomly as the reading of each individual molecule may be analyzed independently from the others. Any other known methods for attaching nucleic acids may be used.

### Detection

Any detection method may be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple tag complement regions.

The present invention provides for detection of molecules from a single nucleotide to a single target nucleic acid molecule. A number of methods are available for this purpose. Methods for visualizing single molecules within nucleic acids labeled with an intercalating dye include, for example, fluorescence microscopy. For example, the fluorescent spectrum and lifetime of a single molecule excited-state can be measured. Standard detectors such as a photomultiplier tube or avalanche photodiode can be used. Full field imaging with a two-stage image intensified COD camera also can be used. Additionally, low noise cooled CCD can also be used to detect single fluorescent molecules.

The detection system for the signal may depend upon the labeling moiety used, which can be defined by the chemistry available. For optical signals, a combination of an optical fiber or charged couple device (CCD) can be used in the detection step. In those circumstances where the substrate is itself transparent to the radiation used, it is possible to have an incident light beam pass through the substrate with the detector located opposite the substrate from the target nucleic acid. For electromagnetic labeling moieties, various forms of spectroscopy systems can be used. Various physical orientations for the detection system are available and discussion of important design parameters is provided in the art.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. It is sometimes referred to as a high-efficiency photon detection system. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. In other words, the optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the immobilized target nucleic acid-primer complex in the presence of a polymerase. TIR fluorescence microscopy is then used to visualize the immobilized target nucleic acid-primer complex and/or the incorporated nucleotides with single molecule resolution.

Measured signals can be analyzed manually or by appropriate computer methods to tabulate results. The substrates and reaction conditions can include appropriate controls for verifying the integrity of hybridization and extension conditions, and for providing standard curves for quantification, if desired. For example, a control primer can be added to the nucleic acid sample for extending a target nucleic acid that is known to be present in the sample (or a target nucleic acid sequence that is added to the sample). The absence of the expected extension product is an indication that there is a defect with the sample or assay components requiring correction.

Exemplary methods and devices for preparing a surface of a substrate for immobilizing a target nucleic acid are provided in Examples 1-12 of U.S. patent application Ser. No. 11/137,928, filed May 25, 2005, the teachings of which are incorporated herein in their entirety. Nucleotides particularly useful in the invention comprise detectable labels. Labeled nucleotides include any nucleotide that has been modified to include a label that is directly or indirectly detectable. Preferred labels include optically-detectable labels, including fluorescent labels or fluorophores, such as fluorescein, rhodamine, cyanine, cyanine-5 dye, cyanine-3 dye, or a derivative or modification of any of the foregoing, and also include such labeling systems as hapten labeling. Accordingly, methods of the invention further provide for exposing the primer/target nucleic acid complex to a digoxigenin, a fluorescein, an alkaline phosphatase or a peroxidase.

In one embodiment, fluorescence resonance energy transfer (FRET) as a detection scheme. FRET in the context of sequencing is described generally in Braslavasky, et al., Sequence Information can be Obtained from Single DNA Molecules, Proc. Nat'l Acad. Sci., 100: 3960-3964 (2003), incorporated by reference herein. Essentially, in one embodiment, a donor fluorophore is attached to the primer, polymerase, or template. Nucleotides added for incorporation into the primer comprise an acceptor fluorophore that is activated by the donor when the two are in proximity. Activation of the acceptor causes it to emit a characteristic wavelength of light. In this way, incorporation of a nucleotide in the primer sequence is detected by detection of acceptor emission. Of course, nucleotides labeled with a donor fluorophore also are useful in methods of the invention; FRET-based methods of the invention only require that a donor and acceptor fluorophore pair are used, a labeled nucleotide may comprise one fluorophore and either the template or the polymerase may comprise the other. Such labeling techniques result in a coincident fluorescent emission of the labels of the nucleotide and the labeled template or polymerase, or alternatively, the fluorescent emission of only one of the labels.

The present invention also provides devices for automated detection of small RNA molecules in a biological sample. The device comprises a series of functional compartments, including an extractor, whereby RNA can be extracted from a biological sample, a fractionator, whereby the RNA is fractionated by size, a modification chamber, whereby the fractionated RNA can be modified with an adaptor, an attachment chamber, whereby the modified small RNA molecule can be attached to a surface wherein individual small RNA molecules are positioned on the surface such that individual small RNA molecules are individually optically resolvable, and a sequencing chamber, whereby at least one nucleotide of at least one attached modified small RNA molecule can be identified. The extractor, fractionator, and chambers are operably linked to allowed automated detection of small RNA molecules in a biological sample. One or more of the extractor, fractionator, and chambers can perform one or more tasks such as extracting, fractionating, modifying, attaching, or sequencing, wherein the reagents for the particular step are flowed into the compartment with or without washing steps in between.

The present invention also provides combination articles of manufacture comprising an adaptor for modifying small RNA molecules obtained from a biological sample and a surface, whereby small RNA molecules modified with the adaptor can be attached, such that individual modified RNA molecules are individually optically resolvable.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

We claim:

1. A method for detecting a small RNA molecule in a biological sample comprising the steps of:
modifying a small RNA molecule contained in the biological sample with an adaptor;
attaching the modified small RNA molecule to a surface wherein individual small RNA molecules are positioned on the surface such that individual small RNA molecules are individually optically resolvable; and
analyzing the attached modified small RNA molecule, wherein at least one nucleotide is identified in at least one attached modified small RNA molecule,
thereby detecting a small RNA molecule in a biological sample.

2. The method of claim 1, wherein the biological sample comprises total RNA.

3. The method of claim 2, further comprising purifying small RNA molecules from the total RNA comprising the steps of:
separating the total RNA by size; and
obtaining RNA corresponding to about 10 to about 200 nucleotides in length from the separated RNA.

4. The method of claim 1, wherein the adaptor is an oligonucleotide.

5. The method of claim 4, wherein the oligonucleotide comprises an amino group at a 5′ terminus.

6. The method of claim 4, wherein the oligonucleotide comprises a poly(A) sequence.

7. The method of claim 4, wherein the small RNA molecule is modified by ligating the adaptor to the small RNA molecule.

8. The method of claim 4, wherein the small RNA molecule is modified by extending the small RNA molecule with an enzyme.

9. The method of claim 8, wherein the enzyme is yeast poly(A) polymerase.

10. The method of claim 8 further comprising treating the modified small RNA molecule such that an amino group is added to a 5′ terminus of the modified small RNA molecule.

11. The method of claim 1, wherein the modified small RNA molecule is attached to the surface by chemical coupling.

12. The method of claim 11, wherein the modified small RNA molecule comprises an amine group at a 5′ terminus and the surface comprises and epoxide group.

13. The method of claim 1, wherein the modified small RNA molecule is attached to the surface by a binding pair selected from the group consisting of an antigen-antibody binding pair, a biotin-streptavidin binding pair, a digoxigenin-anti-digoxigenin binding pair, photoactivated coupling molecules, and a pair of complementary nucleic acids.

14. The method of claim 4, wherein the modified small RNA molecule is attached to a surface by hybridizing the modified small RNA molecule to a primer, the primer being coupled to the surface and the primer being complementary to a portion of the oligonucleotide sequence sufficient to attach the modified small RNA molecule to the surface, thereby producing a hybridized primer.

15. The method of claim 14 wherein analyzing the sequence comprises:
introducing a labeled nucleotide and a polymerase under conditions that allow template-dependent incorporation of the nucleotide into the hybridized primer; and
determining whether the nucleotide is incorporated into the hybridized primer.

16. The method of claim 15, further comprising repeating steps d) and e) at least once in order to determine a sequence of at least one small RNA molecule.

17. The method of claim 4, wherein analyzing the sequence comprises:
contacting the attached modified small RNA molecule to a primer, the primer being complementary to a portion of the specific sequence oligonucleotide sufficient to hybridize to the specific sequence oligonucleotide, producing a hybridized primer;
introducing a labeled nucleotide and a polymerase under conditions that allow incorporation of the nucleotide into the hybridized primer; and
determining whether the nucleotide is incorporated into the hybridized primer.

18. The method of claim 17, further comprising repeating steps e) and f) at least once in order to determine a sequence of at least one small RNA molecules.

19. The method of claim 1, wherein the number of small RNA molecules in the biological sample having different nucleotide sequences is determined.

20. The method of claim 1, further comprising treating the surface to remove surface defects.

21. A method for detecting a small RNA molecule in a biological sample comprising the steps of:
extracting RNA from a biological sample;
separating the RNA by size;
obtaining RNA corresponding to about 10 to about 200 nucleotides in length from the separated RNA;
modifying the RNA obtained in c) with an adaptor;
attaching the modified RNA to a surface wherein individual RNA molecules are positioned on the surface such that individual small RNA molecules are individually optically resolvable; and
analyzing the sequence of the attached modified RNA molecules, wherein at least one nucleotide is identified in at least one attached modified RNA molecule,
thereby detecting a small RNA molecule in a biological sample.

22. An apparatus for automated detection of small RNA molecules in a biological sample comprising:
an extractor, whereby RNA can be extracted from a biological sample;
a fractionator, whereby the RNA is fractionated by size;
a modification chamber, whereby the fractionated RNA can be modified with an adaptor;
an attachment chamber, whereby the modified RNA can be attached to a surface wherein individual RNA molecules are positioned on the surface such that at least two of the individual RNA molecules are individually optically resolvable; and
a sequencing chamber, whereby at least one nucleotide of at least one attached modified RNA can be identified,
wherein the extractor, fractionator, and chambers are operably linked to allow automated detection of RNA in a biological sample.

23. A combination article of manufacture comprising:
an adaptor for modifying small RNA molecules contained in a biological sample; and
a surface whereby small RNA molecules modified with the adaptor of a) can be attached to the surface wherein individual small RNA molecules are positioned on the surface such that at least two of the individual RNA molecules are individually optically resolvable.

24. A method for sequencing a small RNA, the method comprising the steps of:
obtaining a single-stranded RNA comprising between about 19 and about 27 nucleotides in length;
hybridizing said single-stranded RNA to a surface-bound primer nucleic acid comprising a nucleotide sequence that is complementary to at least a portion of said single-stranded rNA, thereby forming a duplex that is individually optically resolvable;
exposing said duplex to an RNA polymerase and at least one nucleotide under conditions that support template-dependent nucleotide addition to said primer;
determining whether a nucleotide is added to said primer; and
repeating said exposing and determining steps.

25. A method for sequencing a small RNA molecule, the method comprising the steps of:
obtaining a single-stranded RNA comprising between about 19 and about 27 nucleotides in length;
preparing a cDNA complement of said RNA;
hybridizing said cDNA to a surface-bound primer nucleic acid comprising a nucleotide sequence that is complementary to at least a portion of said cDNA, thereby forming a duplex that is individually optically resolvable;
exposing said duplex to a polymerase and at least one nucleotide under conditions that support template-dependent nucleotide addition to said primer;
retermining whether a nucleotide is added to said primer; and
repeating said exposing and determining steps.
